(19) United States
(12) Patent Application Publication
(10) Pub. No.: US 2006/0198904 A1
(43) Pub. Date: Sep. 7, 2006

(54) Methods for inhibiting allergic inflammation and other responses initiated by pollens, molds, and other non-animal derived allergens

(76) Inventors: Istvan Boldogh, Galveston, TX (US); Sanjiv Sur, Houston, TX (US)

Correspondence Address: Peter Rogalskyj;Rogalskyj & Weyand, P.O. Box 44, Livonia, NY, 14487-0044, US

(21) Appl. No.: 10/535,874

(22) PCT Filed: Nov. 20, 2003
(86) PCT No.: PCT/US03/37118

§ 371 Date: Apr. 17, 2006

Related U.S. Application Data
(60) Provisional application No. 60/427,826, filed on Nov. 20, 2002.

(51) Int. Cl. A61K 36/18; A61K 31/375; A61K 31/353; A61K 31/198; A61K 31/355

(52) U.S. Cl. 424/725; 514/456; 514/458; 514/474; 514/562

(57) ABSTRACT

Disclosed are methods for treating or preventing allergic inflammation in a subject and methods for treating or preventing one or more symptoms of allergic inflammation in a subject. The methods include administering to the subject a compound which inhibits reactive oxygen species. Also disclosed are methods for reducing a non-animal-derived allergen's ability to initiate an allergenic process and for reducing prooxidant activity of a non-animal-derived allergen. The methods include contacting the non-animal-derived allergen with an agent which inhibits reactive oxygen species. Methods for screening a compound for its usefulness as an agent for reducing a non-animal-derived allergen's ability to initiate an allergenic process and/or for its usefulness as a therapeutic agent for treating or preventing allergic inflammation and/or the symptoms associated therewith are also disclosed.

The present application claims the benefit of U.S. Provisional Patent Application Ser. No. 60/427,826, filed Nov. 20, 2002, which provisional patent application is hereby incorporated by reference.

The present invention was made with the support of the National Institutes of Health Grant Nos. ES06676, P01AI46004, and CA84461. The Federal Government may have certain rights in this invention.

### FIELD OF THE INVENTION

The present invention is directed, generally, to methods for inhibiting allergic inflammation and, more particularly, to methods for inhibiting allergic inflammation and other responses initiated by pollens, molds, and other non-animal derived allergens.

### BACKGROUND OF THE INVENTION

Allergic inflammation is a major and growing medical problem in the United States and elsewhere. For example, more than ten million persons in the United States suffer from asthma and related inflammatory lung diseases, and the numbers of persons with asthma is increasing both in the United States and worldwide. The morbidity associated with asthma makes asthma a major medical condition. Asthma is the most common chronic disease of childhood and the leading cause, among chronic illnesses, of school absences. Asthma in humans results in an estimated 27 million patient visits, 6 million lost workdays, and 90.5 million days of restricted activity per year. In addition to its morbidity, the mortality rate for asthma is growing worldwide. Additionally, asthma reactions are a growing problem for animals. For example, the horse racing industry is affected by horses that suffer from asthmatic reactions.

In view of the above, a need exists for methods which inhibit allergic inflammation and other responses initiated by allergens. The present invention is directed, in part, to addressing this need.

### SUMMARY OF THE INVENTION

The present invention relates to a method for treating or preventing allergic inflammation in a subject. The method includes administering to the subject a compound which inhibits reactive oxygen species.

The present invention also relates to a method for treating or preventing one or more symptoms of allergic inflammation in a subject. The method includes administering to the subject a compound which inhibits reactive oxygen species.

The present invention also relates to a method for reducing a non-animal-derived allergen's ability to initiate an allergenic process. The method includes contacting the non-animal-derived allergen with an agent which inhibits reactive oxygen species.

The present invention also relates to a method for reducing pro-oxidant activity of a non-animal-derived allergen. The method includes contacting the non-animal-derived allergen with an agent which inhibits reactive oxygen species.

The present invention also relates to a method for screening a compound for its usefulness as an agent for reducing a non-animal-derived allergen's ability to initiate an allergenic process and/or for its usefulness as a therapeutic agent for treating or preventing allergic inflammation and/or the symptoms associated therewith. The method includes evaluating the compound's ability to inhibit reactive oxygen species.

### BRIEF DESCRIPTION OF THE DRAWINGS

FIG. 1A is a bar graph showing reduction of nitroblue tetrazolium (“NBT”) by various allergenic extracts. FIG. 1B is a graph of absorbance as a function of time showing the kinetics of superoxide anion generation. FIG. 1C is a Western blot showing the detection of NADPH oxidase in various allergenic extracts. FIGS. 1D and 1E are images of immunolocalization experiments of NADPH oxidase in ragweed pollen. FIG. 1F is an image showing a hypotonic solution inducing the release of respirable particles from ragweed pollen (upper panel) and an image of a Western blot showing that these particles contain Amb 1 a. FIG. 1G is an image of a PAGE gel showing the results of an in situ NBT assay of various allergenic extracts.

FIG. 2A is a bar graph showing, inter alia, that RWE increases reactive oxygen species (“ROS”) levels in cultured cells. FIG. 2B is a bar graph showing, inter alia, that pretreatment of epithelial cells with N-acetyl-L-cysteine (“NAC”) inhibits RWE-induced increase in intracellular ROS levels. FIGS. 2C and 2D are bar graphs showing that intrapulmoanary challenge of mice with RWE induces various markers of oxidative stress in bronchoalveolar lavage (“BAL”) fluids. FIG. 2E is a compilation of images showing changes in ROS levels in lung epithelium of mice after ex vivo challenge. FIG. 2F is a bar graph showing the effect of intranasal administration of antioxidants (ascorbic acid (“AA”) plus NAC) transiently increases total antioxidant potential in airways.

FIGS. 3A and 3B are bar graphs showing that ROS reconstitute the ability of RWE^{H} and Amb a 1 to induce robust inflammation. FIG. 3C is a bar graph showing that RWE^{H} and Amb a 1 induce similar levels of Th2 cytokines from cultured splenocytes of RWE-sensitized mice. FIGS. 3D and 3E are bar graphs showing that pRWE weakly induces accumulation of eosinophils (FIG. 3D) and total inflammatory cells (FIG. 3E). FIG. 3F is an image of two denaturing polyacrylamide gels showing the protein components of RWE and pRWE.

FIG. 4A is a compilation of images showing that ROS increases antigen-induced accumulation of eosinophils, inflammatory cells and formation of mucous cells. FIG. 4B is a bar graph showing morphometric quantiation of eosinophils in peribronchial lung tissue. FIG. 4C is a compilation of images showing that co-administration of hypoxanthine plus xanthine oxidase with Amb a 1 increases the number of mucous cells in airway epithelium.

FIGS. 5A and 5B are bar graphs showing that antioxidants inhibit accumulation of eosinophils (FIG. 5A) and total inflammatory cells (FIG. 5B) in BAL fluids of RWE-challenged mice. FIGS. 5C and 5D are images showing that AA+NAC inhibits RWE-induced peribronchial recruitment of eosinophils. FIG. 5E is a bar graph showing that delayed administration of antioxidants fails to block RWE-induced lung inflammation.

FIG. 6 is a schematic diagram depicting a two signal ROS antigen model of allergic lung inflammation.

### DETAILED DESCRIPTION OF THE INVENTION

The present invention relates to a method for treating or preventing allergic inflammation in a subject. The method includes administering to the subject a compound which inhibits reactive oxygen species.

“Allergic inflammation”, as used herein, is meant to include any inflammation in a subject which is caused, directly or indirectly, in whole or in part, by exposure of the subject to a non-animal-derived allergen. “Allergen”, as used herein is meant to include allergens as well as isoallergens, major allergens (those in which greater than 50% of those tested have the corresponding allergen-specific IgEs) as well as minor allergens (those in which less than 50% of those tested have the corresponding allergen-specific IgEs), for example as described in the Bulletin of the World Health Organization, 72(5):796-806 (1994), which is hereby incorporated by reference. “Non-animal-derived allergen”, as used herein, is meant to include pollens (e.g., tree pollens, grass pollens, weed pollens, as well as sub-pollen particles, non-IgE-producing molecules and those which may be associated with a change in intracellular levels of reactive oxygen/nitrogen species), molds (e.g., mold spores, mold cells, and/or components containing IgE-inducing and non-inducing molecules and those which may be related to a change in intracellular levels of reactive oxygen/nitrogen species), as well as other allergens which are of non-animal origin. Illustratively, “allergic inflammation” is meant to include allergic inflammation of various mucosal surfaces, such as airway epithelium, mucus membranes in nose, eye, and gastrointestinal lumen (GI allergies such as food allergies), and skin and is meant to include conditions such as asthma, Chronic Obstructive Pulmonary Disease (“COPD”), seasonal allergic airway inflammation, allergic rhinitis, allergic conjunctivitis, gastrointestinal allergies (such as food allergies), and atopic dermatitis. As indicated above, “allergic inflammation” may be caused directly by exposure to the non-animal-derived allergen (as in the case where exposure to the non-animal-derived allergen elicits an immediate hypersensitivity and/or inflammatory response. Alternatively, “allergic inflammation” may be caused indirectly by exposure to the non-animal-derived allergen, for example, as in the case where exposure to the non-animal-derived allergen causes structural or chemical changes in the subject's tissues which make an inflammatory response more likely upon subsequent exposure to any allergen, such as a non-animal-derived allergen (e.g., a pollen or mold), an animal-derived allergen (e.g., animal dander), or a non-biological allergens (e.g., diesel exhaust, pollutants, smoke, air driven particles and other particulate matter, soot, nitrogen dioxide, ozone, other chemicals, etc.).

“Preventing allergic inflammation”, as used herein, is meant to include any form of prevention of an inflammatory response, for example, as determined by the presence and/or numbers of eosinophils and/or other inflammatory cells, presence and/or numbers of mucin producing cells, volume of secreted mucus, airway narrowing, and/or cytokine/chemokine levels and/or changes in serum antibody levels or integrated ventilatory function measurements.

“Treating allergic inflammation”, as used herein, is meant to refer to any reversal of an inflammatory response (e.g., by at least about 5%, such as by at least about 10%, by at least about 20%, by at least about 30%, by at least about 40%, and/or by at least about 50%) or reduction in the progression of an inflammatory response (e.g., by at least about 5%, such as by at least about 10%, by at least about 20%, by at least about 30%, by at least about 40%, and/or by at least about 50%), as determined by any test acceptable to those skilled in the art. Illustratively, the extent of an inflammatory response can be determined by assessing the extent to which eosinophils and other inflammatory cells are present; by assessing the extent to which mucin producing cells are present; by assessing volume of secreted mucus; by assessing airway narrowing; and/or by assessing cytokine/chemokine levels and/or changes in serum antibody levels or integrated ventilatory function.

“Subject”, as used herein, is meant to refer to any organism susceptible to allergic inflammation. Illustratively, subjects are meant to include mammals, such as humans and other primates; rabbits; cats and other felines; dogs and other canines; cows, cattle, and other bovines; pigs and other porcines; horses and other equines; and rats, mice, and other rodents. As further illustration, subjects are meant to include humans who have been diagnosed as having asthma, COPD, and/or seasonal allergic airway inflammation; humans who have not been diagnosed as having asthma, COPD, and/or seasonal allergic airway inflammation; humans who have been diagnosed as having allergic rhinitis; humans who have not been diagnosed as having allergic rhinitis; humans who have been diagnosed as having allergic conjunctivitis; humans who have not been diagnosed as having allergic conjunctivitis; humans who have been diagnosed as having gastrointestinal allergies; humans who have not been diagnosed as having gastrointestinal allergies; humans who have been diagnosed as having atopic dermatitis; and/or humans who have not been diagnosed as having atopic dermatitis. As still further illustration, subjects are meant to include equines who have been diagnosed as suffering from asthma, as well as equines who have not been diagnosed as suffering from asthma.

As discussed above, the method includes administering to the subject a compound which inhibits reactive oxygen species.

As used herein, “reactive oxygen species” is any chemical species containing a reactive oxygen moiety (e.g., an oxygen radical, an oxygen ion, a peroxy moiety, etc.), such as molecules containing oxygen that are in altered chemical states, thereby making them capable of initiating and/or altering cellular activation cascades, oxidatively damage molecules (proteins, lipids, and DNA) of cells and/or tissues. Illustrative examples of “reactive oxygen species” include superoxide radicals, peroxide-containing moieties, hydrogen peroxide, lipid peroxyls, lipid alkoxyls, hypochlorous acid, singlet oxygen, ozone, and the like.

As used herein, a compound is to be deemed to be one which “inhibits reactive oxygen species” if the compound inhibits the formation of the reactive oxygen species, or if the compound inhibits the activity of the reactive oxygen species, or both.

For example, the compound can be a compound which inhibits the formation of reactive oxygen species. One class of such compounds is NADH/NADPH oxidase inhibitors. As used herein, “NADH/NADPH oxidase” is meant to include reduced nicotinamide dinucleotide (“NADH”) oxidases and reduced nicotinamide dinucleotide phosphate (“NADPH”) oxidases, and “NADH/NADPH oxidase inhibitors”, as used herein are meant to include inhibitors of NADH oxidases and/or inhibitors of NADPH oxidases. Illustrative NADH/NADPH oxidase inhibitors include (i) NADH/NADPH oxidase inhibitors which are selective for NADH/NADPH oxidases, (ii) NADH/NADPH oxidase inhibitors which are selective for non-animal NADH/NADPH oxidases, and/or (iii) NADH/NADPH oxidase inhibitors which are selective for pollen and/or mold NADH/NADPH oxidases. In this regard, an inhibitor is deemed to be selective for a particular class of enzymes if it substantially inhibits the activity of enzymes in the class but does not substantially inhibit the activity of enzymes not in the class. Suitable NADH/NADPH oxidase inhibitors include, for example, diphenylene iodonium, apocynin, tiron (4,5-dihydroxy-1,3-benzenedisulfonic acid and salts thereof), pyridine, imidazole, and quinacrine. Another class of compounds which inhibit the formation of reactive oxygen species is those compounds which inhibit the formation of hydrogen peroxide from superoxide moieties (e.g., superoxide radical anions). These include superoxide dismutase inhibitors, such as diethyldithiocarbubamate, disulfiram, indomethacin (a cyclooxygenase-2 inhibitor), and 2-methoxyestradiol.

Alternatively, the compound can be one which inhibits the activity of the reactive oxygen species, for example, by binding to the reactive oxygen species, by quenching the reactive oxygen species' oxygen radical or other reactive oxygen moiety, and/or by destroying the reactive oxygen species. Illustrative of such compounds are scavengers of reactive oxygen species, such as superoxide radical scavengers and hydrogen peroxide radical scavengers. Such compounds can be naturally occurring, or they can be synthetic. Examples of such compounds include tiron, ascorbic acid, tocopherol, 6-hydroxy-2,5,7,8-tetramethylchroman-2-carboxylic acid (TROLOX™), N-acetyl-L-cysteine, and combinations thereof. Other examples of such compounds include plant antioxidants, such as flavonoids, flavonols, flavones, flavanols, isoflavones, antocyanidins (e.g., quercetin, kaempferol, myricetin, apigenin, and luteolin), carotenoids, and polyphenols.

The compound which inhibits reactive oxygen species can be administered to the subject by any suitable route, either systemically or locally.

Illustratively, the compound can be administered locally to the site or sites where allergic inflammation is to be prevented or treated. For example, where the allergic inflammation is asthma or COPD or other allergic inflammation of the subject's airway epithelium (e.g., in the subject's bronchial passages), administration can be carried out by administering the compound to the subject's airway, for example, as an inhaled aerosol or mist delivered via the mouth or via the nose. Where the allergic inflammation is allergic rhinitis or other allergic inflammation of the subject's nasal mucus membranes, administration can be carried out by administering the compound to the subject's nasal cavity, for example, as an inhaled aerosol or mist delivered via the nose or as a topical salve applied to the nasal cavity. Where the allergic inflammation is allergic conjunctivitis or other allergic inflammation of the subject's eye, administration can be carried out by administering the compound to the subject's eye, for example, as a liquid solution or suspension applied, for example, as drops to the eye. Where the allergic inflammation is allergic inflammation of the mucus membranes of the subject's gastrointestinal lumen, administration can be carried out by administering the compound to the subject's gastrointestinal tract, for example, as a liquid solution or suspension, capsule, or tablet. Where the allergic inflammation is atopic dermatitis or other allergic inflammation of the subject's skin, administration can be carried out by topically administering the compound to the subject's skin, for example, as a liquid solution or suspension or as a salve.

Irrespective of whether the compound is administered for therapeutic or preventative purposes, it will be appreciated that the actual preferred effective amount of compound will vary according to the compound employed, the particular composition formulated, and the mode of administration. Many factors that can modify the compound's activity will be taken into account by those skilled in the art; e.g., species, body weight, sex, diet, time of administration, route of administration, rate of excretion, condition of the subject, and reaction sensitivities and severities.

Illustratively, the compound can be administered in a single daily dose, or in multiple doses, or even continuously. Continuous administration can be carried out, for example, using a slow-release suspension, patch, or other formulation. Although the compound can be administered by any mode of systemic drug administration, including oral administration or parenteral (e.g. intradermal, intraventricular, intracerebral, intramuscular, intravenous, intraperitoneal, rectal, and subcutaneous) administration, local administration is likely to be a preferred route.

The compound can be administered alone or in combination with suitable pharmaceutical carriers or diluents. The diluent or carrier ingredients should be selected so that they do not diminish the therapeutic or preventative effects of the compound.

Pharmaceutical and ophthalmological formulations of the present invention can be prepared according to conventional formulating techniques. The pharmaceutical and ophthalmological formulations can include any suitable pharmacologically acceptable carrier or adjuvant, selected, for example, based on the dosage form of the preparation and the route of administration.

For example, for topical dosage forms to be applied to the eye, eye drop formulations can include buffering agents to ensure that the formulation is isotonic. This typically involves adjusting the acidity or alkalinity of the formulation so that it has the same or similar pH to mammalian eye fluids. pH values of between 6.1 to 6.3 are suitable. Various buffering agents can be employed in this regard, such as one or more of zinc sulfate, boric acid, and potassium bicarbonate. Typically, the total amount of buffering agents present in the composition ranges from 1 to 15% by weight. The eye drop composition can also include a lubricant, such as carboxymethyl cellulose or other cellulose derivatives. When used, the lubricant can be present, for example, in an amount of 0.01 to 5% by weight of the composition. The composition can also include a preservative, such as benzalkonium chloride and/or other quaternary ammonium preservative agents, phenylmercuric salts, sorbic acid, chlorobutanol, disodium edetate, thimerosal, methyl and propyl paraben, benzyl alcohol, and phenyl ethanol. When used, the preservative can be present, for example, in an amount of 0.1 to 5% by weight of the composition. The eye drop formulation typically includes a vehicle, such as deionized water or mixtures of water and water-miscible solvents (e.g., lower alkanols or arylalkanols), phosphate buffer vehicle systems, isotonic vehicles such as boric acid, sodium chloride, sodium citrate, sodium acetate, and the like, vegetable oils, polyalkylene glycols, and petroleum based jelly, as well as aqueous solutions containing ethyl cellulose, carboxymethyl cellulose and derivatives thereof, hydroxypropylmethyl cellulose, hydroxyethyl cellulose, carbopol, polyvinyl alcohol, polyvinyl pyrrolidone, isopropyl myristate, and other conventionally-employed non-toxic, pharmaceutically acceptable organic and inorganic carriers. The eye drop formulation may also contain non-toxic auxiliary substances such as emulsifying agents, wetting agents, bodying agents, and the like, such as, for example, polyethylene glycols, carbowaxes, and polysorbate 80. Other conventional ingredients can be employed, such as sorbitan monolaurate, triethanolamine, oleate, polyoxyethylene sorbitan 35 monopalmitylate, dioctyl sodium sulfosuccinate, monothioglycerol, thiosorbitol, ethylenediamine tetraacetic acid, and the like. The eye drop formulation can be applied to the eye in any suitable amount, for example, 1 to 8 drops per day. Application of eye drop formulation to the eye can be carried out once a day or in multiple, substantially equal doses (e.g., 2 to 4 times per day).

Suitable dosage forms for oral use include tablets, dispersible powders, granules, capsules, suspensions, syrups, and elixirs. Inert diluents and carriers for tablets include, for example, calcium carbonate, sodium carbonate, lactose, and talc. Tablets may also contain granulating and disintegrating agents, such as starch and alginic acid; binding agents, such as starch, gelatin, and acacia; and lubricating agents, such as magnesium stearate, stearic acid, and talc. Tablets may be uncoated or may be coated by known techniques to delay disintegration and absorption. Inert diluents and carriers which may be used in capsules include, for example, calcium carbonate, calcium phosphate, and kaolin. Suspensions, syrups, and elixirs may contain conventional excipients, such as methyl cellulose, tragacanth, sodium alginate; wetting agents, such as lecithin and polyoxyethylene stearate; and preservatives, such as ethyl-p-hydroxybenzoate.

Suitable dosage forms for pulmonary delivery to the subject's airways can be in solid or liquid particulate form. Solid or liquid particulate forms of the compound prepared for practicing the present invention include particles of respirable size: that is, particles of a size sufficiently small to pass through the mouth and larynx upon inhalation and into the bronchi and alveoli of the lungs. In general, particles ranging from about 1 to 10 microns in size are within the respirable range. The formulation containing the compound is preferably administered by direct inhalation into the respiratory system for delivery as a mist, or other aerosol, or dry powder. Depending upon the solubility of the particular formulation of compound administered, the daily dose may be divided among one or several unit dose administrations. The doses of the compounds may be provided as one or several prepackaged units. In the manufacture of a dosage form for pulmonary delivery, the compound is typically admixed with, inter alia, an acceptable carrier. The carrier must, of course, be acceptable in the sense of being compatible with any other ingredients in the formulation and must not be deleterious to the patient. The carrier may be a solid, or a liquid, or both and is preferably formulated with the compound as a unit dose formulation. Aerosols of liquid particles comprising the compound may be produced by any suitable means, such as inhalatory delivery systems. One is a traditional nebulizer which works in a mechanism similar to the familiar perfume atomizer. The airborne particles are generated by a jet of air from either a compressor or compressed gas cylinder passing through the device (pressure driven aerosol nebulizer). In addition, other forms utilize an ultrasonic nebulizer by vibrating the liquid at speed of up to about 1 MHz, for example as described in U.S. Pat. No. 4,501,729, which is hereby incorporated by reference. Nebulizers are commercially available devices which transform solutions or suspensions of the active ingredient into a therapeutic aerosol mist either by means of acceleration of compressed gas, typically air or oxygen, through a narrow venturi orifice or by means of ultrasonic agitation. Suitable formulations for use in nebulizers contain the compound in a liquid carrier. The carrier is typically sterile water or a dilute aqueous alcoholic solution, which may be isotonic or hypertonic with body fluids. Optional additives include preservatives, such as methyl hydroxybenzoate, flavoring agents, volatile oils, buffering agents, and surfactants which are normally used in the preparation of pharmaceutical compositions. Aerosols of solid particles comprising the compound may likewise be produced with any solid particulate medicament aerosol generator. Aerosol generators for administering solid particulate medicaments to a subject produce particles which are respirable, as explained above, and generate a volume of aerosol containing a predetermined metered dose of a medicament at a rate suitable for human administration. One illustrative type of solid particulate aerosol generator is an insufflator. Suitable formulations for administration by insufflation include finely comminuted powders which may be delivered by means of an insufflator or taken into the nasal cavity in the manner of a snuff. In the insufflator, the powder is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the compound or of a powder blend which includes the compound, a suitable powder diluent, such as lactose, and an optional surfactant. A second type of illustrative aerosol generator involves us of a metered dose inhaler. Metered dose inhalers are pressurized aerosol dispensers, typically containing a suspension or solution formulation of the compound in a liquified propellant. During use, these devices discharge the formulation through a valve, adapted to deliver a metered volume, e.g., from 10 to 22 microliters to produce a fine particle spray containing the compound. Suitable propellants include certain chlorofluorocarbon compounds, for example, dichlorodifluoromethane, trichlorofluoromethane, dichlorotetrafluoroethane and mixtures thereof. The formulation may additionally contain one or more co-solvents, for example, ethanol, surfactants, such as oleic acid or sorbitan trioleate, and suitable flavoring agents. Any propellant may be used in carrying out the present invention, including both chlorofluorocarbon-containing propellants and non-chlorofluorocarbon-containing propellants. Fluorocarbon aerosol propellants that may be employed in carrying out the present invention include fluorocarbon propellants in which all hydrogens are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Examples of such propellants include, but are not limited to: CF₃CHFCF₂, CF₃CH₂CF₂H, CF₃CHFCF₃, CF₃CH₂CF₃, CF₃CHClCF₂Cl, CF₃CHClCF₃, CF₃CHClCH₂Cl, CF₃CHFCF₂Cl, and the like. A stabilizer such as a fluoropolymer may optionally be included in formulations of fluorocarbon propellants, such as described in U.S. Pat. No. 5,376,359, which is hereby incorporated by reference. Compositions containing respirable dry particles of micronized compound may be prepared by grinding the dry compound with, e.g., a mortar and pestle or other appropriate grinding device, and then passing the micronized composition through a 400 mesh screen to break up or separate out large agglomerates. The aerosol, whether formed from solid or liquid particles, may be produced by the aerosol generator at a rate of from about 10 to about 150 liters per minute. Aerosols containing greater amounts of compound may be administered more rapidly. Typically, each aerosol may be delivered to the patient for a period from about 30 seconds to about 20 minutes, with a delivery period of about 1 to about 5 minutes being preferred. The particulate composition comprising the compound may optionally contain a carrier which serves to facilitate the formation of an aerosol. A suitable carrier is lactose, which may be blended with the active compound in any suitable ratio. In addition to the compound and carriers and excipients mentioned above, dosage forms for pulmonary delivery may further include other pharmacologically active materials, such as bronchodilators.

Suitable dosage forms for topical application to the skin include salves, creams, lotions, topical sprays, solutions, suspensions, and emulsions. For example, the compound can be formulated in a dermatologically acceptable vehicle compatible with the skin, such as corn oil, aqueous ethanol, isopropanol, sesame oil, propylene glycol, benzyl alcohol, oleyl alcohol, isopropyl esters of fatty acids, such as myristic and palmitic acids, mineral oil, and/or wax. The vehicle can be of such a viscosity and/or wetting power that the composition may be satisfactorily applied to the skin as a continuous film or coating. The amount of compound present in the topical formulation is not particularly critical. Illustratively, the topical formulation can contain from about 0.5 to 10% by weight of the compound. The topical formulation can be applied to the skin once a day, or multiple applications can be used (e.g., 2 to 4 times per day).

Suitable dosage forms for delivery of compound to the nasal cavity can include the compound and a non-toxic pharmaceutically acceptable nasal carrier. Suitable non-toxic pharmaceutically acceptable nasal carriers for use in the methods of the present invention will be apparent to those skilled in the art of nasal pharmaceutical formulations. Obviously the choice of suitable carriers will depend on the exact nature of the particular nasal dosage form desired, as well as on the identity of the compound. For example, the compounds can be formulated into a nasal solution (for use as drops or spray), a nasal suspension, a nasal ointment, or a nasal gel. The nasal solutions, suspensions, and gels can contain, in addition to the compound, a major amount of water (preferably purified water) and minor amounts of other ingredients such as pH adjusters (e.g., a base such as NaOH), emulsifiers or dispersing agents (e.g. polyoxyethylene 20 sorbitan mono-oleate), buffering agents, preservatives, wetting agents, and gelling agents (e.g. methylcellulose). Also, a sustained release composition (e.g., a sustained release gel) can be readily prepared. The above-mentioned liquid nasal formulations (e.g., solutions or suspensions) can be administered as drops, sprays, or by any other liquid intranasal dosage form. Optionally, the delivery system can be a unit dose delivery system. The volume of solution or suspension delivered per dose can be anywhere from about 5 to 400 microliters, such as from about 50 to about 150 microliters. Delivery systems for these various dosage forms can be dropper bottles, plastic squeeze units, atomizers, and the like, in either unit dose or multiple dose packages.

Dosage forms suitable for parenteral administration include solutions, suspensions, dispersions, emulsions, microcapsules and the like. They may also be manufactured in the form of sterile solid compositions which can be dissolved or suspended in sterile injectable medium immediately before use. They may contain suspending or dispersing agents known in the art. Where microcapsules are employed, they can be readily prepared by conventional microencapsulation techniques, such as those disclosed in, for example, Encyclopedia of Chemical Technology, 3rd edition, Volume 15, New York: John Wiley and Sons, pp. 470-493 (1981), which is hereby incorporated by reference.

As discussed above, administration can be carried out in a single dose, in multiple doses, or even continuously. In one embodiment, administration is commenced subsequent to the subject's being exposed to a pollen, mold, or other non-animal-derived allergen, for example, as in the case where administration is commenced within about 1 day, such as within about 18 hours, within about 12 hours, within about 10 hours, within about 8 hours, within about 6 hours, within about 4 hours, within about 2 hours, within about 1 hour of the subject's exposure to a pollen, mold, or other non-animal-derived allergen. Where multiple-dose or continuous administration is employed, administration can be carried out for a suitable period of time, such as for from about 10 minutes to about 1 week, such as from about 30 minutes to about 2 days, from about 1 hour to about 1 day, etc.) after it is commenced. Additionally or alternatively, administration can be carried out prior to the time that the subject is exposed to a pollen, mold, or other non-animal-derived allergen (for example, up to about 12 hours, such as up to about 6 hours, up to about 4 hours, up to about 2 hours, up to about 1 hour, up to about 30 minutes, up to about 20 minutes, up to about 10 minutes, and/or up to about 5 minutes prior to exposure or expected exposure to pollen, mold, or other non-animal-derived allergen, depending on the route of administration, the rate at which the compound is expelled or metabolized, etc.). In such cases, administration can be complete at the time that the subject is exposed to a pollen, mold, or other non-animal-derived allergen; or administration can be ongoing at the time that the subject is exposed to a pollen, mold, or other non-animal-derived allergen; and, in either case, administration can be re-commenced or continued subsequent to the subject's being exposed to a pollen, mold, or other non-animal-derived allergen, for example, as described above.

The present invention, in another aspect thereof, relates to a method for treating or preventing one or more symptoms of allergic inflammation in a subject. The method includes administering to the subject a compound which inhibits reactive oxygen species.

Symptoms of allergic inflammation include, for example, rhinorrhea, sneezing, nasal pruritus, congestion, coughing, chest tightness, shortness of breath, wheezing, flaky patches of dry skin, redness of the eyes, and/or watering of the eyes. Illustratively, for asthma, symptoms can include cough, chest tightness, and wheezing, caused, for example, by inflammation and excessive irritability of bronchi (main air passages in the lung); for anaphylaxis (a severe allergic reaction), symptoms can include hives, swelling in the throat, difficulty breathing, wheezing, fainting, and loss of blood pressure; and, for allergic rhinitis (hay fever), symptoms can include nasal stuffiness, runny nose, sneezing, itching in the nose and throat, itchy, watery, and/or red eyes, fatigue, and headache. These and other symptoms of allergic inflammation can be treated or prevented by administering to the subject a compound which inhibits reactive oxygen species, for example, as described hereinabove.

The present invention also relates to a method for reducing a non-animal-derived allergen's ability to initiate an allergenic process. The method includes contacting the pollen, mold, or other non-animal-derived allergen with an agent which inhibits reactive oxygen species.

“Agent”, as used herein, is meant to include compounds which inhibit the formation of the reactive oxygen species, or which inhibit the activity of the reactive oxygen species, or both, as well as physical agents which inhibit the formation of the reactive oxygen species, or which inhibit the activity of the reactive oxygen species, or both. Suitable compounds which inhibit the formation of the reactive oxygen species or which inhibit the activity of the reactive oxygen species include those discussed hereinabove. Such compounds include NADH/NADPH oxidase inhibitors, such as NADH/NADPH oxidase inhibitors which are selective for NADH/NADPH oxidases, NADH/NADPH oxidase inhibitors which are selective for non-animal NADH/NADPH oxidases, and/or NADH/NADPH oxidase inhibitors which are selective for pollen and/or mold NADH/NADPH oxidases; diphenylene iodonium; apocynin; tiron; pyridine; imidazole; quinacrine; inhibitors of hydrogen peroxide formation; superoxide dismutase inhibitors; diethyldithiocarbamate; disulfiram; indomethacin; 2-methoxyestradiol; compounds which inhibit the activity of the reactive oxygen species; scavengers of reactive oxygen species; superoxide radical scavengers; hydrogen peroxide scavengers; ascorbic acid; tocopherol; 6-hydroxy-2,5,7,8-tetramethylchroman-2-carboxylic acid (TROLOX™); N-acetyl-L-cysteine; plant antioxidants, such as flavonoids, flavonols, flavones, flavanols, isoflavones, antocyanidins (e.g., quercetin, kaempferol, myricetin, apigenin, and luteolin), carotenoids, and polyphenols; and/or combinations thereof. Suitable physical agents which inhibit the formation of the reactive oxygen species or which inhibit the activity of the reactive oxygen species include, for example, heat, particularly an amount of heat which is sufficient to substantially reduce (e.g., by at least about 30%, such as by at least about 40%, by at least about 50%, by at least about 60%, by at least about 70%, by at least about 80%, by at least about 85%, by at least about 90%, and/or by at least about 95%) the NADH/NADPH oxidase activity of the pollen, mold, or other non-animal-derived allergen. Illustratively, suitable heat treatments include heating the non-animal-derived allergen at a temperature from about 60° C. to about 90° C. for a period of time from about 15 minutes to about 1 hour, such as heating the non-animal-derived allergen at a temperature from at least about 70° C. to about 90° C. for a period of time of from at least about 15 minutes to about 1 hour, heating the non-animal-derived allergen at a temperature from at least about 72° C. to about 90° C. for a period of time from at least about 15 minutes to about 1 hour, heating the non-animal-derived allergen at a temperature from at least about 72° C. to about 90° C. for a period of time from at least about 30 minutes to about 1 hour, and/or heating the non-animal-derived allergen at a temperature from at least about 72° C. to about 80° C. for a period of time of at least about 30 minutes.

Contacting can be carried out with a compound in vivo, for example, by administering the compound to a subject in a manner described hereinabove. Alternatively, contacting can be carried out ex vivo, e.g., at a time prior to the non-animal-derived allergen's coming into contact with the subject. For example, such ex vivo contact can be carried out by spraying a composition containing the compound on the non-animal-derived allergen or by drawing the non-animal-derived allergen through a polymeric, paper, or other mesh (e.g., a vacuum cleaner bag, a furnace filter, a air-purification filter, a dust mask, etc.) into or onto which the compound has been applied.

The present invention also relates to a method for reducing pro-oxidant activity of a non-animal-derived allergen. The method includes contacting the pollen, mold, or other non-animal-derived allergen with an agent which inhibits reactive oxygen species.

Suitable agents for use in this aspect of the present invention include compounds which inhibit the formation of the reactive oxygen species, or which inhibit the activity of the reactive oxygen species, or both, as well as physical agents which inhibit the formation of the reactive oxygen species, or which inhibit the activity of the reactive oxygen species, or both. Suitable compounds which inhibit the formation of the reactive oxygen species or which inhibit the activity of the reactive oxygen species include those discussed hereinabove. Such compounds include NADH/NADPH oxidase inhibitors, such as NADH/NADPH oxidase inhibitors which are selective for NADH/NADPH oxidases, NADH/NADPH oxidase inhibitors which are selective for non-animal NADH/NADPH oxidases, and/or NADH/NADPH oxidase inhibitors which are selective for pollen and/or mold NADH/NADPH oxidases; diphenylene iodonium; apocynin; tiron; pyridine; imidazole; quinacrine; inhibitors of hydrogen peroxide formation; superoxide dismutase inhibitors; diethyldithiocarbamate; disulfiram; indomethacin; 2-methoxyestradiol; compounds which inhibit the activity of the reactive oxygen species; scavengers of reactive oxygen species; superoxide radical scavengers; hydrogen peroxide radical scavengers; ascorbic acid; tocopherol; 6-hydroxy-2,5,7,8-tetramethylchroman-2-carboxylic acid (TROLOX™); N-acetyl-L-cysteine; plant antioxidants, such as flavonoids, flavonols, flavones, flavanols, isoflavones, antocyanidins (e.g., quercetin, kaempferol, myricetin, apigenin, and luteolin), carotenoids, and polyphenols; and/or combinations thereof. Suitable physical agents which inhibit the formation of the reactive oxygen species or which inhibit the activity of the reactive oxygen species include, for example, heat, particularly an amount of heat which is sufficient to substantially reduce (e.g., by at least about 30%, such as by at least about 40%, by at least about 50%, by at least about 60%, by at least about 70%, by at least about 80%, by at least about 85%, by at least about 90%, and/or by at least about 95%) the NADH/NADPH oxidase activity of the pollen, mold, or other non-animal-derived allergen. Suitable heat treatments include those described hereinabove.

Contacting can be carried out with a compound in vivo, for example, by administering the compound to a subject in a manner described hereinabove. Alternatively, contacting can be carried out ex vivo, e.g., at a time prior to the non-animal-derived allergen's coming into contact with the subject. For example, such ex vivo contact can be carried out by spraying a composition containing the compound on the non-animal-derived allergen or by drawing the non-animal-derived allergen through a polymeric, paper, or other mesh (e.g., a vacuum cleaner bag, a furnace filter, a air-purification filter, a dust mask, etc.) into or onto which the compound has been applied.

The present invention, in yet another aspect thereof, relates to a method for screening a compound for its usefulness as an agent for reducing a non-animal-derived allergen's ability to initiate an allergenic process and/or for its usefulness as a therapeutic agent for treating or preventing allergic inflammation and/or the symptoms associated therewith. The method includes evaluating the compound's ability to inhibit reactive oxygen species. Illustratively, the method can be carried out by evaluating the compound's ability to inhibit the formation of reactive oxygen species, such as by evaluating the compound's ability to inhibit NADH/NADPH oxidase, by evaluating the compound's ability to inhibit pollen and/or mold NADH/NADPH oxidases, and/or by evaluating the compound's ability to inhibit superoxide dismutase. Alternatively, the method can be carried out by evaluating the compound's ability to inhibit the activity of reactive oxygen species, such as by evaluating the compound's ability to scavenge reactive oxygen species, by evaluating the compound's ability to scavenge superoxide radicals, and/or by evaluating the compound's ability to scavenge hydrogen peroxide radicals.

In one such method, the compound to be screened is added to a solution or suspension of a NADH/NADPH oxidase in the presence of nitroblue tetrazolium (“NBT”), and the conversion of NBT to formazan is spectrophotometrically monitored, for example at 530 nm. Reduced conversion of NBT to formazan (as indicated, for example, by an increase in NBT absorbance at 530 nm) indicates that the screened compound is an inhibitor of the NADH/NADPH oxidase, thus capable of inhibiting the formation of reactive oxygen species, and thus a candidate for use as an agent for reducing a non-animal-derived allergen's ability to initiate an allergenic process and/or for use as a therapeutic agent for treating or preventing allergic inflammation and/or the symptoms associated therewith.

In another such method, the screened compound's ability to scavenge reactive oxygen species can be evaluated using conventional methods, such as those described in Naguib, “A Fluorometric Method for Measurement of Peroxyl Radical Scavenging Activities of Lipophilic Antioxidants,” Anal. Biochem., 265:290-298 (1998) and DeLange et al., “Phycoerythrin Fluorescence-Based Assay for Peroxy Radicals: A Screen for Biologically Relevant Protective Agents,” Anal. Biochem., 177:300-306 (1989), which are hereby incorporated by reference.

The screening method of the present invention can further include administering the test compound to a population of subjects and recording adverse side-effects of the test compound. For example, such further screening can be undertaken as part of a clinical study designed specifically to assess the test compound's toxicity (using, for example, animal models); or it can be undertaken as part of a clinical study designed primarily to assess the test compound's efficacy (for example, in humans); or it can be part of ongoing reporting requirements associated with regulatory approval (even after the test compound has been granted regulatory approval).

Compounds identified as having the ability to inhibit reactive oxygen species in accordance with the method of the present invention can be used to reduce a non-animal-derived allergen's ability to initiate an allergenic process and/or to treat or prevent allergic inflammation and/or the symptoms associated therewith, for example using the methods described hereinabove.

Certain aspects of the present invention are further illustrated with the following examples.

### EXAMPLES

### Example 1

### Overview of the Studies Described in Examples 2-6

In this Example 1 and in the following Examples 2-6, numerals in square brackets refer to references that are set forth in Example 7. Each of these references is hereby incorporated by reference.

Asthma is an inflammatory disease of the airways that is associated with oxidative stress generated by inflammatory cells recruited to the airways after exposure to allergens [1-4]. An unresolved question is whether enzymatic activities in pollens directly induce oxidative stress prior to inflammation. Plant cells have NADPH oxidases that are similar to the inducible NADPH oxidase complex in the plasma membrane of mammalian phagocytes [5-7]. These plant oxidases play a critical role in vital physiological functions, including defense against pathogens [6,8], growth and development [7,9]. A recent study has demonstrated that reactive oxygen species (“ROS”) produced by NADPH oxidases regulate expansion of cells in root hairs [10]. Pollens have many enzymatic activities [11], but the presence of NADPH oxidases has not previously been reported. Since pollen germination and formation of the pollen tubes are likely to require ROS, we sought to determine whether pollen grains contain NADPH oxidases and, if so, to delineate their role in induction of oxidative stress and allergic inflammation in the airways.

### Example 2

### All Tested Pollens Possess Intrinsic NADPH Oxidase Activity

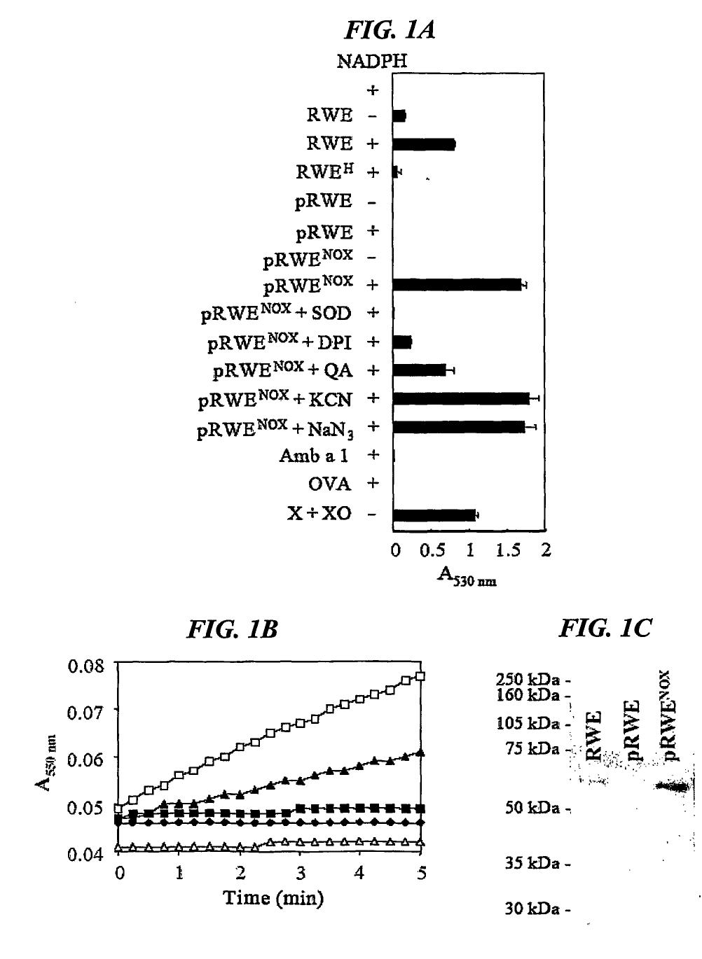

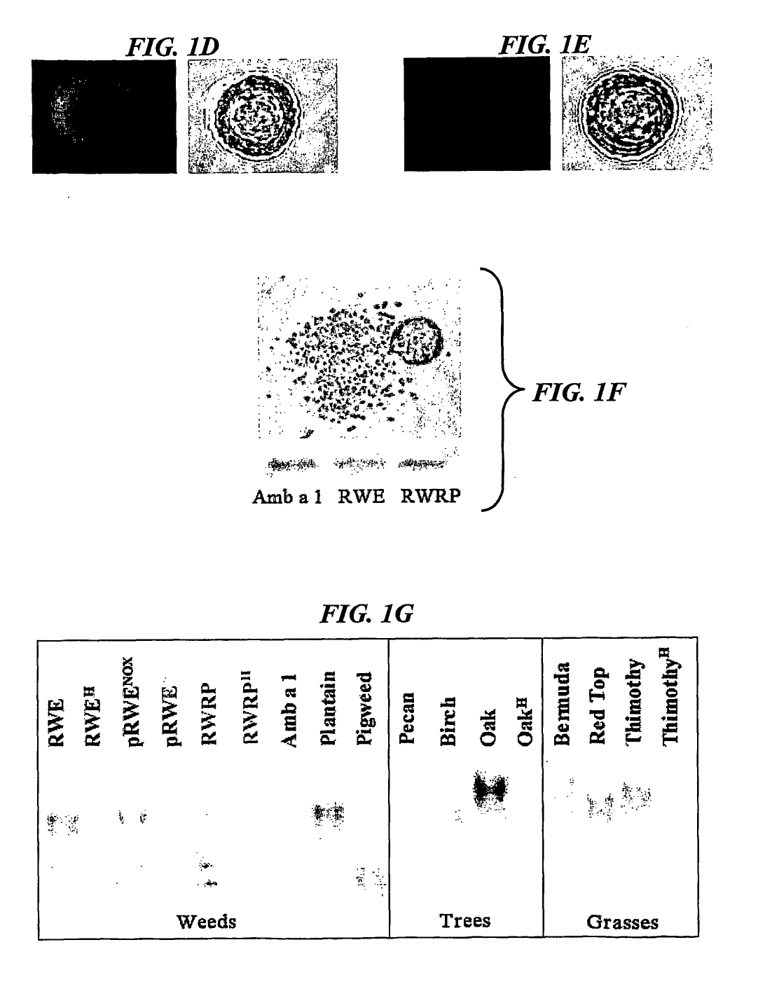

We show here that ragweed (Ambrosia artemisiifolia) pollen extract (“RWE”) effectively reduces nitroblue tetrazolium (“NBT”) to formazan (FIG. 1A), suggesting that it generates superoxide anions (O₂.⁻). Adding NADPH to RWE boosted NBT reduction (FIG. 1A), implying that RWE contains NADPH dependent oxidases. To accurately quantify formazan in an NBT assay, we separated pigments from RWE by Superdex 200 filtration. Because filtration also separates substrates from enzymes, none of the pigment-free fractions reduced NBT. Adding NADPH to the fractions reconstituted the ability of some fractions to reduce NBT. These NBT-reactive (pRWE^{NOX}) and the NBT non-reactive (pRWE) fractions were pooled separately. The NBT reducing activity of pRWE^{NOX} was inhibited by NADPH oxidase inhibitors diphenyleneiodonium and quinacrine [12], but was resistant to the respiratory inhibitors cyanide and azide [7] (FIG. 1A). These observations suggest that the enzyme in pRWE^{NOX} that reduces NBT is NADPH oxidase(s) but not peroxidase [7]. We used a cytochrome c assay [13] to confirm the superoxide-producing activity of pRWE^{NOX}. The mixture of pRWE^{NOX}, ferricytochrome c, and NADPH, but not these reagents separately, increased the absorbance (A) at 550 nm, and this increase was abrogated by superoxide dismutase (“SOD”) [13], confirming production of superoxide anions (O₂.⁻) (FIG. 1B). In parallel experiments, a decrease in A_{340 nm} [14] indicated that pRWE^{NOX} utilized NADPH (data not shown), corroborating that the enzyme activity present in RWE and pRWE^{NOX} is NADPH oxidase. Heat treatment (72° C. for 30 min) of RWE (RWE^{H}) and pRWE^{NOX} abrogated their NADPH oxidase activity. Amb a 1, the major antigen in RWE [15], did not possess this activity. Because antibodies raised against components of human NADPH oxidase cross-react with plant NADPH oxidase-related proteins [16,17], we used an antibody against human p67^{phox} to perform Western blotting and immunohistochemistry to further confirm the presence of NADPH oxidases in pollens. Western blot analysis identified a single band in RWE and pRWE^{NOX}, but not in pRWE (FIG. 1C). This antibody reacted with the periplasmic region of ragweed pollen grains (FIGS. 1D and 1E). Recent studies have shown that exposure of pollen grains to hypotonic solution such as rain water rapidly induces formation of respirable particles (0.2 to 3 μm) [18,19]. Consistent with these observations, adding water to ragweed pollen grains induced release of respirable particles (RWRP, FIG. 1F). In situ NBT reduction assay [20] and Western blot analysis demonstrated that these particles have NADPH oxidase activity (FIG. 1G) and contain Amb a 1 (FIG. 1F). These findings indicate that NADPH oxidase in ragweed pollen grains and respirable particles are present in RWE.

We examined whether extracts of other pollens have NADPH oxidase activity by an in situ NBT reduction assay [20]. Immersing the gel in a buffer containing NADPH induced formation of well-defined NBT-reducing protein bands in lanes with RWE, pRWE^{NOX}, RWRP, and pollen extracts of trees, grasses, and weeds (FIG. 1G). Amb a 1, pRWE, and heat-treated extracts of ragweed, oak, and timothy pollens failed to induce NBT-reactive bands (FIG. 1G). These findings indicate that allergenic extracts from grass, tree, and weed pollens contain NADPH oxidases.

### Example 3

### NADPH Oxidases Intrinsic to Pollens Induce Oxidative Stress in Cultured Cells and Lungs

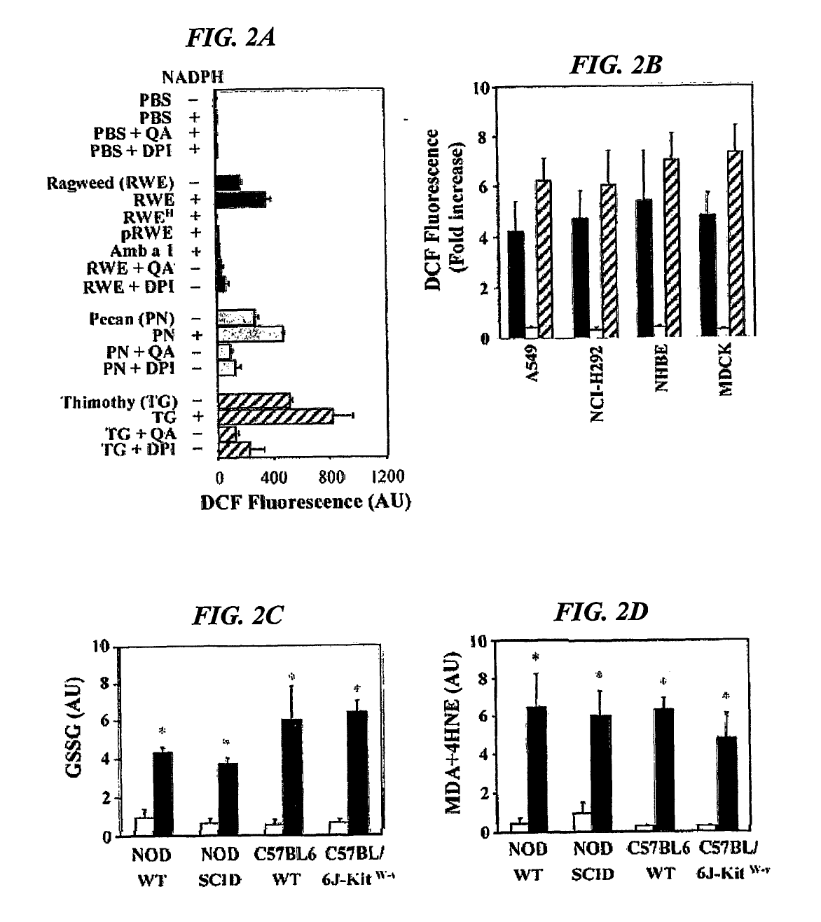

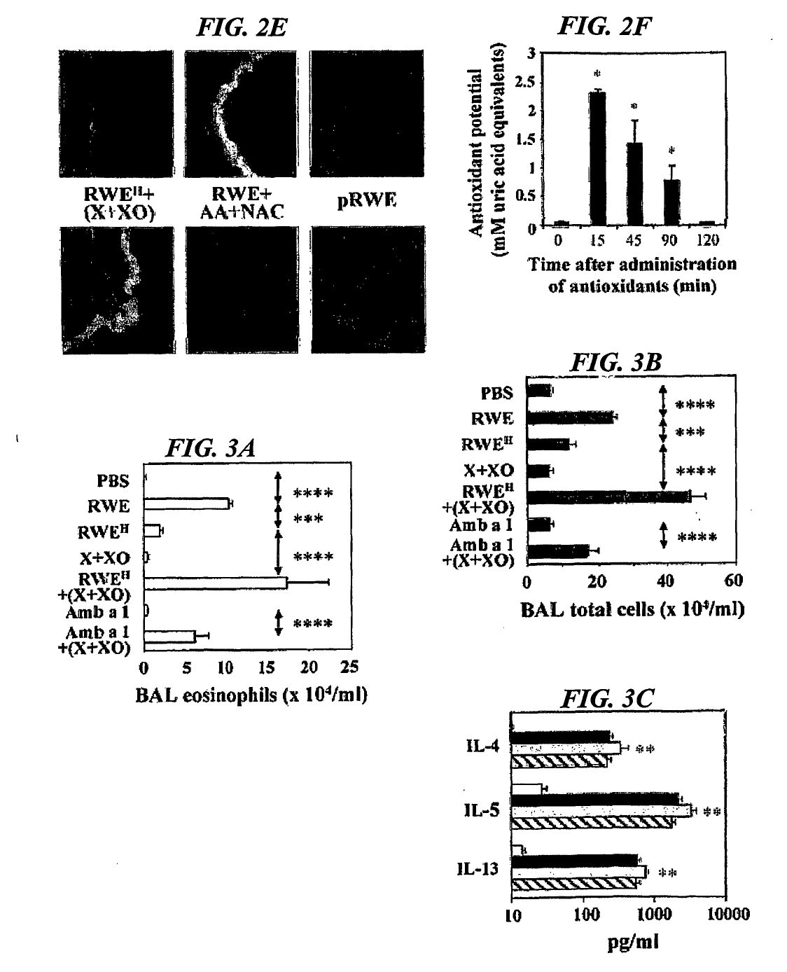

Next, we examined the effects of pollen extracts on cultured epithelial cells and in the lungs of mice. RWE, pecan tree and timothy grass pollen extracts all converted the intracellular redox-sensitive 2′,7′-dihydro-dichlorofluorescein diacetate (H₂DCF-DA) in confluent, cultured normal human bronchial epithelial (“NHBE”) cells into fluorescent dichlorofluorescin (FIG. 2A). RWE also increased intracellular ROS levels in NHBE cells grown in air-liquid interface [21] (data not shown) and in other lung-(NCI-H292) and kidney-(MDCK) derived epithelial cells (FIG. 2B). Elevation in ROS levels was inhibited by NADPH oxidase inhibitors diphenyleneiodonium and quinacrine [12] (FIG. 2A), and also by pre-treating cells with the antioxidant N-acetyl-L-cysteine (“NAC”) [22] (FIG. 2B). Consistent with their lack of NADPH oxidase activity, RWE^{H}, pRWE, and Amb a 1 failed to change the cellular redox state (FIG. 2A).

To elucidate whether pollen NADPH oxidases generate ROS in the lungs of mice, we performed intrapulmonary challenge with PBS (vehicle) or RWE [23-25]. RWE increased the levels of oxidative stress markers in bronchoalveolar lavage (“BAL”) fluids within 15 min after challenge, including oxidized glutathione (GSSG), malondialdehyde, and 4-hydroxynonenal (FIGS. 2C and 2D). RWE, but not extracts that lack NADPH oxidase activity (RWE^{H} and pRWE), enhanced ROS levels in airway epithelium of challenged mice within 15 min (FIG. 2E). Co-administration of ROS scavengers ascorbic acid (“AA”) and NAC [26,27] with RWE blocked the increase in RWE-induced epithelial ROS levels (FIG. 2E). This is consistent with our observation that antioxidants administered by this route transiently increase the total antioxidant potential of airway lining fluid (FIG. 2F). Because an adaptive immune response is believed to be critical for ROS production in the airways [3,28], we examined the effects of RWE challenge in mice deficient in mast cells (C57BL/6J-Kit^{W-v}) [29], and in mice deficient in B-cells, T-cells and immunoglobulins (NOD SCID, a non-leaky mouse strain with severe combined immunodeficiency) [30]. Challenge with RWE induced the same levels of oxidative stress markers in knockout mice as in wild-type mice (FIGS. 2C and 2D). These data indicate that the NADPH oxidases of RWE rapidly increase ROS levels in the airways independent of adaptive immunity, and this is prevented by co-administering ROS scavengers.

### Example 4

### Pollen Antigens and NADPH Oxidase Activity are both Required for Inducing Robust Allergic Luna Inflammation

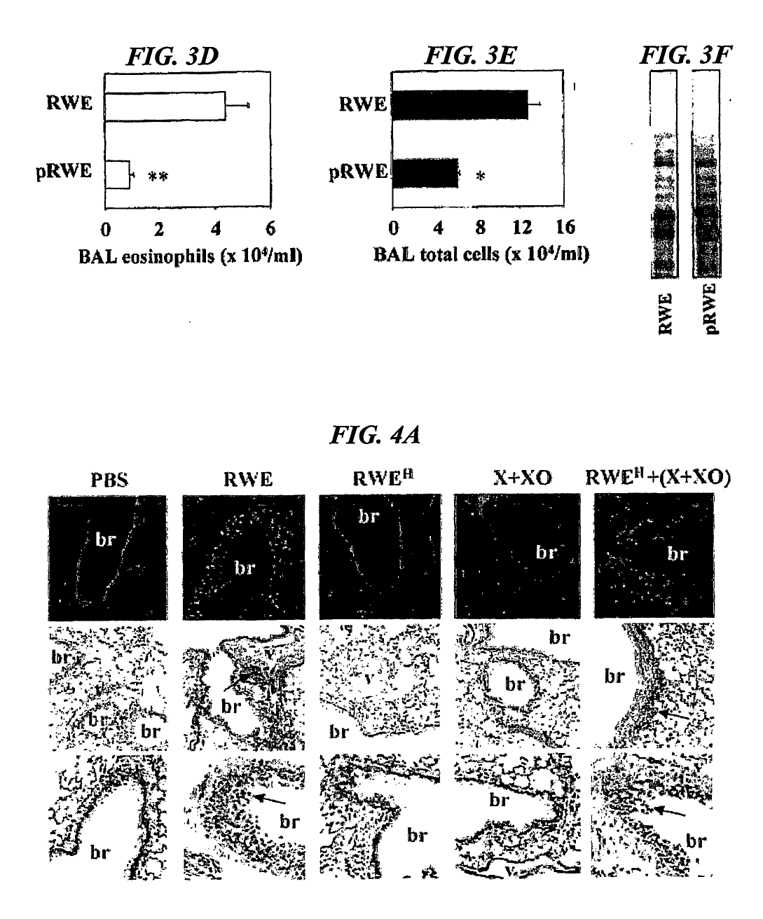

We investigated whether ROS generated by pollen NADPH oxidases augment airway inflammation in murine asthma. Consistent with our previous publications [25,31], challenge with RWE vigorously induced accumulation of eosinophils (FIG. 3A) and total inflammatory cells (FIG. 3B) in the BAL fluid (airway compartment). However, challenge with extracts that lack NADPH oxidase activity (RWE^{H} or Amb a 1) caused significantly less accumulation of eosinophils and other inflammatory cells in the BAL fluids (FIGS. 3A and 3B). Amb a 1 induced similar levels of T-helper 2 (Th2) cytokines (e.g., IL-4, IL-5, IL-13) from cultured splenocytes, as did RWE, whereas RWE^{H} induced more cytokines than RWE (FIG. 3C). These findings indicate that the inability of RWE^{H} and Amb a 1 to induce airway inflammation is not due to an inability to stimulate Th2 cells. We hypothesize that the failure of RWE^{H} and Amb a 1 to induce allergic inflammation was due to absence of O₂.⁻ generation by pollen NADPH oxidases (FIG. 1B). Consistent with this hypothesis, administration of surrogate O₂.⁻ generator hypoxanthine (X) plus xanthine oxidase (XO) with either RWE^{H} or Amb a 1 significantly increased the accumulation of inflammatory cells (FIGS. 3A and B). To validate the importance of NADPH oxidase activity of RWE in the accumulation of inflammatory cells, we performed intrapulmonary challenge with equal quantities of RWE and pRWE (which lacks NADPH oxidase activity) in parallel groups of RWE-sensitized mice. Even though pRWE has a similar protein profile as RWE (SDS-PAGE analyses, FIG. 3F), challenge with pRWE recruited fewer eosinophils (FIG. 3D) and total inflammatory cells (FIG. 3E) in BAL fluids than did challenge with RWE.

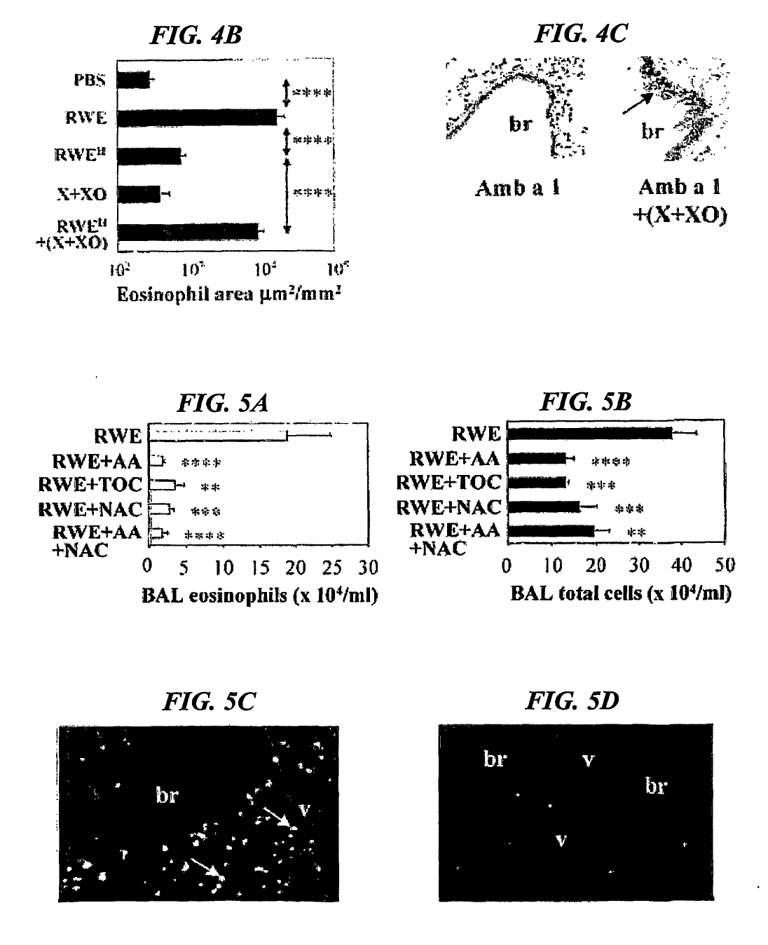

To evaluate the impact of pollen NADPH oxidases on tissue distribution of inflammatory and mucous cells, we compared the effects of intrapulmonary challenge with RWE vs. RWE^{H}. The latter induced significantly less eosinophilic and total inflammation in peribronchial and perivascular locations (FIG. 4A, upper and middle panels, and FIG. 4B) and less mucous cells, consistent with a reduced ability to induce metaplasia of Clara cells to mucous cells [32] (FIG. 4A, lower panel). Likewise, challenge with Amb a 1 failed to increase the number of mucous cells (FIG. 4C). Co-administration of X+XO with RWE^{H} “reconstituted” allergic eosinophilic and peribronchial inflammation (FIG. 4A, upper and middle panels, and FIG. 4B) and increased the number of mucus-containing cells (FIG. 4A, lower panel). Likewise, co-administration of X+XO with Amb a 1 vigorously increased the number of mucous cells (FIG. 4C) in the lungs. However, administration of X+XO by itself failed to induce recruitment of inflammatory cells or formation of mucous cells (FIGS. 4A, 4B, and 4C), indicating that O₂.⁻ generation by itself does not provide sufficient signal to induce these effects. Thus, it is believed that the signal generated by ROS produced by pollen NADPH oxidase(s) acts in concert with the signal induced by pollen antigen(s) to provoke robust allergic airway inflammation.

### Example 5

### Scavenging ROS in the Airways Significantly Reduces Allergic Lung Inflammation

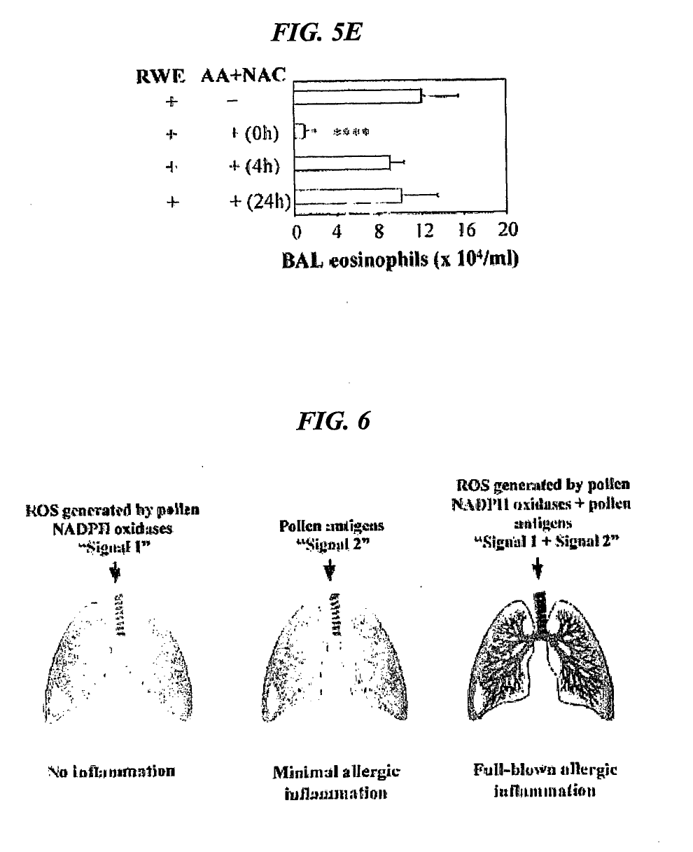

To dissect out the role of NADPH oxidase in RWE in inducing allergic inflammation, we co-administered scavengers of ROS with RWE, either singly or in combination. Administration of antioxidants significantly decreased RWE-induced accumulation of eosinophils and total inflammatory cells in BAL fluids (FIGS. 5A and 5B), and of eosinophils in peribronchial and perivascular locations (FIGS. 5C and 5D). To evaluate which source of ROS is critical for boosting airway inflammation (i.e., ROS generated by the intrinsic NADPH oxidase activity of pollens or ROS generated later by inflammatory cells), we administered AA+NAC, either simultaneously with RWE, or 4 h or 24 h after RWE challenge. Consistent with our prediction, simultaneous administration of AA+NAC with RWE inhibited eosinophils' accumulation, but not when antioxidant mixture was administered 4 h or 24 h after RWE challenge (FIG. 5E). These findings indicate that oxidative stress induced within minutes of RWE challenge, and not that generated by recruited inflammatory cells, plays a critical role in augmenting allergic lung inflammation.

### Example 6

### Discussion

We discovered that NADPH oxidases intrinsic to pollens of weeds, trees, and grasses induce oxidative stress in the lung epithelium within minutes, independent of adaptive immunity. This oxidative insult augments antigen-induced allergic lung inflammation and formation of mucous cells.

In our study, pollen NADPH oxidase rapidly generated O₂.⁻ well before recruitment of inflammatory cells. The airway lining fluid contains extracellular SOD [33], an enzyme that can reduce O₂.⁻ to H₂O₂. The latter could be further reduced to the hydroxyl radical (.OH) in the presence of Fe²⁺. H₂O₂ and .OH can react with lipids to form lipid-hydroperoxides, which may explain the presence of malondialdehyde and 4-hydroxynonenal in BAL minutes after RWE challenge. Airway epithelium and alveolar macrophages secrete extracellular glutathione peroxidase into the epithelial lining fluid [34], and this enzyme eliminates H₂O₂ while converting glutathione into GSSG. This process may explain the elevated levels of GSSG we observe in the airway lining fluid after challenge. Prior studies have indicated that oxidative stress in asthma is induced by recruited inflammatory cells [1-3] and by exposure to oxidant environmental pollutants [35,36]. Because pollen grains and pollen respirable particles penetrate into the intrathoracic airways [18,19,37,38], NADPH oxidases in them are likely to also contribute to oxidative insult to lung epithelium.

We propose a “two-signal ROS-antigen” model of pollen protein-induced initiation of allergic lung inflammation (FIG. 6). In this model, the intrinsic pollen NADPH oxidases generate ROS in epithelial cells that provides “Signal 1”. Presentation of major pollen antigens to Th2 cells generates an antigen-specific “Signal 2” via T-cell recognition. These signals work in concert to induce full-blown allergic airway inflammation. Thus, pollen proteins that do not deliver Signal 1 (Amb a 1, RWE^{H}, and pRWE) are unable to induce a robust allergic lung inflammation. Signal 1 does not have to be specifically induced by NADPH oxidases, because it can be replaced by a surrogate system of ROS generation (X+XO). The immunological specificity of the system comes from Signal 2.

### Example 7

### Methods

Cell Lines. Cells were obtained from the ATCC (A549, NCI-H292 and MDCK), or Cambrex Bio Science (Walkersville, Md.) (NHBE). Cells were maintained in humidified CO₂ (5%) incubator and medium as recommended by the suppliers.

Animals and Sensitization. Animal experiments were performed according to the National Institute of Health Guide for Care and Use of Experimental Animals. Six- to eight-week-old female BALB/c, C57BL/6wild-type, C57BL/6J-Kit^{W-v}, NOD wild-type and NOD SCID mice purchased from Harlan Sprague-Dawley (San Diego, Calif.) or Jackson Laboratory (Bar Harbor, Me.) were used for these studies. Airway inflammation was induced with RWE (Greer Laboratory, N.C.) sensitization and challenges [23,24]. Briefly, mice were sensitized on days 0 and 4 with 150 μg/mouse RWE mixed with alum, as previously described [31]. On day 11, mice were challenged intranasally with RWE (100 μg), RWE^{H} (100 μg) or Amb a 1 (10 μg). Our studies employed intranasal instillation of the antioxidants (all purchased from Sigma-Aldrich, St. Louis, Mo.) NAC (120 mg/kg), AA (130 mg/kg), tocopherol (1,900 U/kg), or their combination. To generate O₂.⁻, xanthine (0.32 mM)+xanthine oxidase (50 mU, Sigma-Aldrich) were mixed with RWE^{H} or Amb a 1 in some groups.

NBT Assay. RWE, pRWE, pRWE^{NOX}, RWE^{H}, Amb a 1, and ovalbumin (“OVA”) (all contained 50 μg protein) were mixed with NBT (2 mM) with or without NADPH (100 μM). Mixtures were then incubated for 15 min at 37° C., and the sedimented formazan was dissolved in methanol. The absorbance was determined at 530 nm in a spectrophotometer (DU 530, Beckman Instruments, Fullerton, Calif.).

Cytochrome c Assay for Detecting O₂.⁻ Generation. The method described by Messner and Imaly was used [13]. The assay was carried out in PBS with 10 μg pRWE and 10 μM oxidized cytochrome c (Sigma-Aldrich) in the reaction mixture. The reactions were started by adding 100 μM NADPH, and the increase in A_{550 nm} was recorded for 5 min. Saturating amounts of SOD 9(50 U/ml, Sigma-Aldrich) were used in a reference reaction to determine any such O₂.⁻ independent reduction of cytochrome c by the sample.

In situ NBT Reduction Assay. 50 μg of extract per lane was electrophoresed on a 6% non-denaturing PAGE at 4° C. The gels were immersed in Tris-glycine buffer (100 mM, pH 7.4) containing NBT (2 mM). Bands appeared within minutes after adding NADPH (1 mM).

Measurements of Reactive Oxygen Species. Epithelial cells (A549, NCI-H292, NHBE, and MDCK) were “loaded” with 50 μM H₂DCF-DA (Molecular Probes, Eugene, Oreg.) at 37° C. for 15 minutes. After washing out excess probe, cells were treated with various allergenic extracts with or without NADPH (100 μM), NAC (1 mM), or NADPH oxidase inhibitors: quinacrine (500 μM) and diphenylene iodonium (100 μM). The change in fluorescence intensity was assessed in a FLx800 micro plate reader (Bio-Tek Instruments, Winooski, Vt.), or by flow cytometry (FACScan, Becton Dickinson, Franklin Lakes, N.J.) [39] at 488 nm excitation and 530 nm emission. Each data point represents the mean fluorescence from three or more independent experiments, and is expressed ±s.e.m.

Airways of mice were “loaded” by intranasal instillation of 100 μl carboxy-H₂DCF-DA (250 μM for 15 min) (Molecular Probes). After washing out excess probe, the mice were sacrificed, and lungs were challenged ex vivo with PBS, or 100 μg of RWE, RWE^{H}, RWE^{H}+(X+XO), RWE+AA+NAC, pRWE in 700 μl volume. These intraluminal challenge materials were removed after 15 min by repeated lavage. The lungs were inflated with and embedded in Tissue-Tek O.C.T. Compound (Sakura Finetek, Torrance, Calif.), frozen, and sectioned. The oxidized product (dichlorofluorescin) of redox-sensitive probe was visualized with a NIKON Eclipse TE 200 UV microscope (excitation at 485 nm). Images were taken with a Photometrix CoolSNAP Fx camera.

Measurement of Oxidative Stress Markers. Mice (NOD and C57BL/6 strains) were challenged with RWE (100 μg), and BAL fluids were collected after 30 minutes, clarified by centrifugation (3,000 rpm for 10 min at 4° C.), and aliquots stored at −80° C. Butylated hydroxytoluene (0.5 mM, Sigma-Aldrich) was added to prevent further lipid oxidation in some aliquots. GSSG levels were measured spectrophotometrically at 412 nm using a GSH/GSSG-412 kit (Bioxytech, OXIS, Portland, Oreg.). Malondialdehyde and 4-hydroxynonenal were quantified using an LPO-586 assay kit (OXIS) and measuring absorbance at 580 nm.

Assay for Total Antioxidant Potential. After intrapulmonary administration of AA and NAC in BALB/c mice, the mice were euthanized at various time points and BAL fluids were collected. The “total antioxidant potential” in BAL samples was measured spectrophotometrically at 490 nm using an AOP-490 assay kit (OXIS). A standard of known uric acid concentration was used to create a calibration curve. The results of the assay were expressed as “mM uric acid equivalents” directly from the plot of absorbance change versus mM uric acid concentration.

Evaluation of Allergic Inflammation. BAL cell counts were assessed by performing total and differential counts on BAL fluid 72 hours after challenge, as we previously described [23,24,40]. For immunohistochemical detection and quantitation of eosinophils [41], cryosections of five different levels of lungs were incubated with rabbit anti-mouse eosinophil major basic protein IgG (anti-MBP) or normal rabbit IgG, and detected with goat anti-rabbit Cy3-conjugated antibody (Jackson ImmunoResearch, West Grove, Pa.). A fully motorized NIKON Eclipse TE 200 UV microscope equipped for inverted fluorescence was used to scan multiple airways and surrounding 0.4 mm of peribronchial area. Images were taken at 10× magnification using Photometrix CoolSNAP Fx digital camera and analyzed by Metamorph software (Version 5, Universal Imaging, Downingtown, Pa.). If the entire area could not be visualized within one frame, several images were obtained and reassembled using the montage stage stitching algorithm of the METAMORPH™ software [42]. The integrated morphometric analysis function was used to transform total pixel area of the fluorescent signal from the eosinophils into μm² units after calibration [43]. The data were represented as area of eosinophils (fluorescent signal) in μm² per mm² of total peribronchial area. The experiment was conducted in three animals of each group (five levels per lung per animal), and the mean is represented here.

Statistics. Data were analyzed by ANOVA, followed by Fishers post-hoc analyses for least significant difference. Differences were considered significant at P<0.05.

### Example 8

### DETAILED DESCRIPTION OF THE FIGURES

FIGS. 1A-1G demonstrate that plant pollen extracts possess NADPH oxidase activity. FIG. 1A shows the reduction of NBT to formazan by allergenic extracts. RWE, pRWE, pRWE^{NOX}, RWE^{H}, Amb a 1, and ovalbumin (“OVA”) were subjected to NBT assay. X+XO was used as a positive control. DPI, diphenyleneiodonium; QA, quinacrine; KCN, potassium cyanide; NaN₃, sodium azide. FIG. 1B shows the kinetics of superoxide anion generation determined by cytochrome c assay. ♦, Cytochrome c (“Cyt c”) alone; ▪, pRWE^{NOX}+Cyt c; Δ, NADPH+Cyt c; □, pRWE^{NOX}+NADPH+Cyt c; ▴, pRWE^{NOX}+NADPH+Cyt c+SOD. FIG. 1C shows detection of NADPH oxidase by Western blot analysis using a rabbit IgG antibody against human p67^{phox} (cat. # sc-15342, Santa Cruz Biotechnology, Calif.). FIGS. 1D and 1E show immunolocalization of NADPH oxidase in ragweed pollens using anti-p67^{phox} antibody (FIG. 1D) and control normal rabbit IgG (FIG. 1E). Right panels are light microscopic images of the same pollens. FIG. 1F shows ragweed pollen respirable particles. The upper panel shows that hypotonic solution induces release of respirable particles from ragweed pollen. The lower panel demonstrates that particles contain Amb a 1 as shown by Western blotting (anti-Amb a 1 antibody, Greer Laboratory). FIG. 1G shows the results of an in situ NBT reduction assay of various weed, tree, and grass pollen samples. Superscripted “H” refers to samples which were heat-inactivated.

FIGS. 2A-2F show that RWE increases ROS levels in cultured epithelial cells and in airway epithelium of mice. FIG. 2A demonstrates, inter alia, that RWE increases ROS levels in cultured NHBE cells. FIG. 2B demonstrates that pretreatment of epithelial cells with NAC inhibits the RWE-induced increase in intracellular ROS levels. Solid bars, RWE; open bars, RWE+NAC; diagonally hatched bars, X+XO. FIGS. 2C and 2D demonstrates that intrapulmoanary challenge of mice with RWE induces markers of oxidative stress GSSG (FIG. 2C) and malondialdehyde (“MDA”) and 4-hydroxynonenal (“4HNE”) (FIG. 2D) in BAL fluids. Open bars, PBS; solid bars, RWE; AU, arbitrary units. Results are means±s.e.m. (n=4-6 mice per group). *P<0.05. FIG. 2E shows changes in ROS levels in lung epithelium of mice after ex vivo challenge. Magnification, 100×. FIG. 2F shows that intranasal administration of antioxidants (AA+NAC) transiently increases total antioxidant potential in airways. *P<0.05.

FIGS. 3A-3F demonstrate that ROS induced by NADPH oxidase augments pollen antigen-induced allergic lung inflammation. FIGS. 3A and 3B show that ROS reconstitute the ability of RWE^{H} and Amb a 1 to induce robust inflammation. Results are means±s.e.m. (n=7-9 mice per group). ***P<0.001; ****P<0.0001. FIG. 3C shows that RWE^{H} and Amb a 1 induce similar levels of Th2 cytokines from cultured splenocytes of RWE-sensitized mice. Open bars, PBS; solid bars, RWE; shaded bars, RWEH; diagonally hatched bars, Amb a 1. Results are means±s.e.m. (n=4-6 independent experiments). **P<0.01. FIGS. 3D and 3E show that pRWE weakly induces accumulation of eosinophils (FIG. 3D) and total inflammatory cells (FIG. 3E). Results are means±s.e.m. (n=5-7 mice per group). *P<0.05; **P<0.01. FIG. 3F show the protein components of RWE (left panel) and pRWE (right panel) in denaturing polyacrylamide gel.

FIGS. 4A-4C demonstrate the impact of intrinsic pollen NADPH oxidases on lung tissue distribution of inflammatory cells and mucous cells. FIG. 4A shows that ROS increases antigen-induced accumulation of eosinophils, inflammatory cells and formation of mucous cells. Upper panel (100× magnification): immunohisto-chemical staining of lung cryosections for eosinophils; middle panel (100× magnification): inflammatory cells in peribronchial and perivascular regions of lungs in formalin-fixed and H&E-stained sections; lower panel (200× magnification): periodic acid-Schiff (“PAS”) staining for mucous cells in lung sections. Magenta-colored epithelial cells are positive for mucin (arrow); br, bronchiole; v, vessel. FIG. 4B shows morphometric quantiation of eosinophils in peribronchial lung tissue. Lung cryosections were immunohistochemically stained as in FIG. 4A, and the number of the eosinophil cells in peribronchial area was quantified. Sections from five levels per lung per animal (n=3) were evaluated and the means±s.e.m. are represented here. ****P<0.0001. FIG. 4C shows that co-administration of X+XO with Amb a 1 increases the number of mucous cells in airway epithelium. Magnification, 100×.

FIGS. 5A-5E demonstrate that scavenging of ROS generated by intrinsic pollen NADPH oxidase inhibits the extent of allergic lung inflammation. FIGS. 5A and 5B show that antioxidants inhibit accumulation of eosinophils (FIG. 5A) and total inflammatory cells (FIG. 5B) in BAL fluids of RWE-challenged mice. TOC, tocopherol. Results are means±s.e.m. (n=7-9 mice per group). **P<0.01; ***P<0.001; **** P<0.0001. FIGS. 5C and 5D show that AA+NAC inhibits RWE-induced peribronchial recruitment of eosinophils (arrows). RWE-sensitized mice were challenged with RWE (FIG. 5C) or RWE plus AA+NAC (FIG. 5D). Magnification: 100×. FIG. 5E shows that delayed administration of antioxidants fails to block RWE-induced lung inflammation. Results are means±s.e.m. (n=7-9 mice per group). ****P<0.0001.

FIG. 6 is a schematic diagram depicting a two signal ROS antigen model of allergic lung inflammation.

### Example 9

### REFERENCES CITED IN EXAMPLES 1-8

1. Dworski, R. Oxidant stress in asthma. Thorax 55 Suppl 2, S51-3 (2000).

2. Calhoun, W. J., Reed, H. E., Moest, D. R. & Stevens, C. A. Enhanced superoxide production by alveolar macrophages and air-space cells, airway inflammation, and alveolar macrophage density changes after segmental antigen bronchoprovocation in allergic subjects. Am Rev Respir Dis 145, 317-25. (1992).

3. Sanders, S. P. et al. Spontaneous oxygen radical production at sites of antigen challenge in allergic subjects. Am J Respir Crit Care Med 151, 1725-33 (1995).

4. Bowler, R. P. & Crapo, J. D. Oxidative stress in allergic respiratory diseases. J Allergy Clin Immunol 110, 349-56 (2002).

5. Doke, N. et al. The oxidative burst protects plants against pathogen attack: mechanism and role as an emergency signal for plant bio-defence—a review. Gene 179, 45-51 (1996).

6. Wojtaszek, P. Oxidative burst: an early plant response to pathogen infection. Biochem J 322 (Pt 3), 681-92 (1997).

7. Frahry, G. & Schopfer, P. NADH-stimulated, cyanide-resistant superoxide production in maize coleoptiles analyzed with a tetrazolium-based assay. Planta 212, 175-83 (2001).

8. Sagi, M. & Fluhr, R. Superoxide production by plant homologues of the gp91(phox) NADPH oxidase. Modulation of activity by calcium and by tobacco mosaic virus infection. Plant Physiol 126, 1281-90 (2001).

9. Schopfer, P., Plachy, C. & Frahry, G. Release of reactive oxygen intermediates (superoxide radicals, hydrogen peroxide, and hydroxyl radicals) and peroxidase in germinating radish seeds controlled by light, gibberellin, and abscisic acid. Plant Physiol 125, 1591-602 (2001).

10. Foreman, J. et al. Reactive oxygen species produced by NADPH oxidase regulate plant cell growth. Nature 422, 442-6 (2003).

11. Bagarozzi, D. A., Jr. & Travis, J. Ragweed pollen proteolytic enzymes: possible roles in allergies and asthma. Phytochemistry 47, 593-8 (1998).

12. Van Gestelen, P., Asard, H. & Caubergs, R. J. Solubilization and Separation of a Plant Plasma Membrane NADPH-O2-Synthase from Other NAD(P)H Oxidoreductases. Plant Physiol 115, 543-550 (1997).

13. Messner, K. R. & Imlay, J. A. In vitro quantitation of biological superoxide and hydrogen peroxide generation. Methods Enzymol 349, 354-61 (2002).

14. Zhang, Z., Yu, J. & Stanton, R. C. A method for determination of pyridine nucleotides using a single extract. Anal Biochem 285, 163-7 (2000).

15. Rafnar, T. et al. Cloning of Amb a I (antigen E), the major allergen family of short ragweed pollen. J Biol Chem 266, 1229-36. (1991).

16. Desikan, R., Hancock, J. T., Coffey, M. J. & Neill, S. J. Generation of active oxygen in elicited cells of Arabidopsis thaliana is mediated by a NADPH oxidase-like enzyme. FEBS Lett 382, 213-7 (1996).

17. Dwyer, S. C., Legendre, L., Low, P. S. & Leto, T. L. Plant and human neutrophil oxidative burst complexes contain immunologically related proteins. Biochim Biophys Acta 1289, 231-7 (1996).

18. Taylor, P. E., Flagan, R. C., Valenta, R. & Glovsky, M. M. Release of allergens as respirable aerosols: A link between grass pollen and asthma. J Allergy Clin Immunol 109, 51-6 (2002).

19. Grote, M., Valenta, R. & Reichelt, R. Abortive pollen germination: A mechanism of allergen release in birch, alder, and hazel revealed by immunogold electron microscopy. J Allergy Clin Immunol 111, 1017-23 (2003).

20. Lopez-Huertas, E., Corpas, F. J., Sandalio, L. M. & Del Rio, L. A. Characterization of membrane polypeptides from pea leaf peroxisomes involved in superoxide radical generation. Biochem J 337 (Pt 3), 531-6 (1999).

21. Booth, B. W., Adler, K. B., Bonner, J. C., Tournier, F. & Martin, L. D. Interleukin-13 induces proliferation of human airway epithelial cells in vitro via a mechanism mediated by transforming growth factor-alpha. Am J Respir Cell Mol Biol 25, 739-43 (2001).

22. van Zandwijk, N. N-acetylcysteine (NAC) and glutathione (GSH): antioxidant and chemopreventive properties, with special reference to lung cancer. J Cell Biochem Suppl 22, 24-32 (1995).

23. Sur, S. et al. Long term prevention of allergic lung inflammation in a mouse model of asthma by CpG oligodeoxynucleotides. J Immunol 162, 6284-93. (1999).

24. Sur, S., Kita, H., Gleich, G. J., Chenier, T. C. & Hunt, L. W. Eosinophil recruitment is associated with IL-5, but not with RANTES, twenty-four hours after allergen challenge. J Allergy Clin Immunol 97, 1272-8. (1996).

25. Wild, J. S. et al. IFN-gamma-inducing factor (IL-18) increases allergic sensitization, serum IgE, Th2 cytokines, and airway eosinophilia in a mouse model of allergic asthma. J Immunol 164, 2701-10 (2000).

26. Som, S., Raha, C. & Chatterjee, I. B. Ascorbic acid: a scavenger of superoxide radical. Acta Vitaminol Enzymol 5, 243-50 (1983).

27. Urban, T., Akerlund, B., Jarstrand, C. & Lindeke, B. Neutrophil function and glutathione-peroxidase (GSH-px) activity in healthy individuals after treatment with N-acetyl-L-cysteine. Biomed Pharmacother 51, 388-90 (1997).

28. Henricks, P. A. & Nijkamp, F. P. Reactive oxygen species as mediators in asthma. Pulm Pharmacol Ther 14, 409-20 (2001).

29. Kawakami, T. & Galli, S. J. Regulation of mast-cell and basophil function and survival by IgE. Nat Rev Immunol 2, 773-86 (2002).

30. Lapidot, T., Fajerman, Y. & Kollet, O. Immune-deficient SCID and NOD/SCID mice models as functional assays for studying normal and malignant human hematopoiesis. J Mol Med 75, 664-73 (1997).

31. Sur, S. et al. Immunomodulatory effects of IL-12 on allergic lung inflammation depend on timing of doses. J Immunol 157, 4173-80 (1996).

32. Reader, J. R. et al. Pathogenesis of mucouscell metaplasia in a murine asthma model. Am J Pathol 162, 2069-78 (2003).

33. Oury, T. D., Day, B. J. & Crapo, J. D. Extracellular superoxide dismutase in vessels and airways of humans and baboons. Free Radic Biol Med 20, 957-65 (1996).

34. Comhair, S. A., Thomassen, M. J. & Erzurum, S. C. Differential induction of extracellular glutathione peroxidase and nitric oxide synthase 2 in airways of healthy individuals exposed to 100% O(2) or cigarette smoke. Am J Respir Cell Mol Biol 23, 350-4 (2000).

35. Depuydt, P. O., Lambrecht, B. N., Joos, G. F. & Pauwels, R. A. Effect of ozone exposure on allergic sensitization and airway inflammation induced by dendritic cells. Clin Exp Allergy 32, 391-6 (2002).

36. Peden, D. B., Setzer, R. W., Jr. & Devlin, R. B. Ozone exposure has both a priming effect on allergen-induced responses and an intrinsic inflammatory action in the nasal airways of perennially allergic asthmatics. Am J Respir Crit Care Med 151, 1336-45 (1995).

37. Driessen, M. N. & Quanjer, P. H. Pollen deposition in intrathoracic airways. Eur Respir J 4, 359-63 (1991).

38. Michel, F. B., Marty, J. P., Quet, L. & Cour, P. Penetration of inhaled pollen into the respiratory tract. Am Rev Respir Dis 115, 609-16 (1977).

39. Ribardo, D. A. et al. Early cell signaling by the cytotoxic enterotoxin of Aeromonas hydrophila in macrophages. Microb Pathog 32, 149-63 (2002).

40. Michalec, L. et al. CCL7 and CXCL10 orchestrate oxidative stress-induced neutrophilic lung inflammation. J Immunol 168, 846-52 (2002).

41. Makela, M. J. et al. The failure of interleukin-10-deficient mice to develop airway hyperresponsiveness is overcome by respiratory syncytial virus infection in allergen-sensitized/challenged mice. Am J Respir Crit Care Med 165, 824-31 (2002).

42. Loo, B. W., Jr., Meyer-Ilse, W. & Rothman, S. S. Automatic image acquisition, calibration and montage assembly for biological X-ray microscopy. J Microsc 197 (Pt 2), 185-201 (2000).

43. Klimaschewski, L., Nindl, W., Pimpl, M., Waltinger, P. & Pfaller, K. Biolistic transfection and morphological analysis of cultured sympathetic neurons. J Neurosci Methods 113, 63-71 (2002).

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

What is claimed is:

1. A method for treating or preventing allergic inflammation in a subject, said method comprising:
administering to the subject a compound which inhibits reactive oxygen species.

2. A method according to claim 1, wherein the compound inhibits the formation of reactive oxygen species.

3. A method according to claim 2, wherein the compound is a NADH/NADPH oxidase inhibitor.

4. A method according to claim 2, wherein the compound is a NADH/NADPH oxidase inhibitor that is selective for NADH/NADPH oxidases.

5. A method according to claim 2, wherein the compound is a NADH/NADPH oxidase inhibitor that is selective for non-animal NADH/NADPH oxidases.

6. A method according to claim 2, wherein the compound is a NADH/NADPH oxidase inhibitor that is selective for pollen and/or mold NADH/NADPH oxidases.

7. A method according to claim 2, wherein the compound is a superoxide dismutase inhibitor.

8. A method according to claim 1, wherein the compound inhibits the activity of reactive oxygen species.

9. A method according to claim 8, wherein the compound is a scavenger of reactive oxygen species.

10. A method according to claim 9, wherein the compound is a superoxide radical scavenger.

11. A method according to claim 9, wherein the compound is a hydrogen peroxide radical scavenger.

12. A method according to claim 8, wherein the compound is a natural compound.

13. A method according to claim 8, wherein the compound is a synthetic compound.

14. A method according to claim 8, wherein the compound is selected from the group consisting of ascorbic acid, tocopherol, 6-hydroxy-2,5,7,8-tetramethylchroman-2-carboxylic acid, N-acetyl-L-cysteine, plant antioxidants, and combinations thereof.

15. A method according to claim 1, wherein the allergic inflammation is an allergic inflammation of the subject's mucosal surface and/or of the subject's skin.

16. A method according to claim 1, wherein the allergic inflammation is an allergic inflammation of the subject's airway epithelium, of mucus membranes in the subject's nose, of mucus membranes in the subject's eye, of mucus membranes in the subject's gastrointestinal lumen, and/or of the subject's skin.

17. A method according to claim 1, wherein said administering is carried out locally.

18. A method according to claim 1, wherein said administering is carried out locally by administering the compound to the subject's eye, skin, and/or gastrointestinal tract.

19. A method according to claim 1, wherein said administering is carried out locally by administering the compound to the subject's airway.

20. A method according to claim 1, wherein said administering is carried out systemically.

21. A method according to claim 1, wherein the subject is a human.

22. A method according to claim 1, wherein the subject is a human who has previously experienced allergic inflammation.

23. A method for treating or preventing one or more symptoms of allergic inflammation in a subject, said method comprising:
administering to the subject a compound which inhibits reactive oxygen species.

24. A method according to claim 23, wherein the compound inhibits the formation of reactive oxygen species.

25. A method according to claim 24, wherein the compound is a NADH/NADPH oxidase inhibitor.

26. A method according to claim 24, wherein the compound is a NADH/NADPH oxidase inhibitor that is selective for NADH/NADPH oxidases.

27. A method according to claim 24, wherein the compound is a NADH/NADPH oxidase inhibitor that is selective for non-animal NADH/NADPH oxidases.

28. A method according to claim 24, wherein the compound is a NADH/NADPH oxidase inhibitor that is selective for pollen and/or mold NADH/NADPH oxidases.

29. A method according to claim 24, wherein the compound is a superoxide dismutase inhibitor.

30. A method according to claim 23, wherein the compound inhibits the activity of reactive oxygen species.

31. A method according to claim 30, wherein the compound is a scavenger of reactive oxygen species.

32. A method according to claim 31, wherein the compound is a superoxide radical scavenger.

33. A method according to claim 31, wherein the compound is a hydrogen peroxide radical scavenger.

34. A method according to claim 30, wherein the compound is a natural compound.

35. A method according to claim 30, wherein the compound is a synthetic compound.

36. A method according to claim 30, wherein the compound is selected from the group consisting of ascorbic acid, tocopherol, 6-hydroxy-2,5,7,8-tetramethylchroman-2-carboxylic acid, N-acetyl-L-cysteine, plant antioxidants, and combinations thereof.

37. A method according to claim 23, wherein the one or more symptoms is caused, in whole or in part, by an allergic inflammation of the subject's mucosal surface and/or of the subject's skin.

38. A method according to claim 23, wherein the one or more symptoms is caused, in whole or in part, by an allergic inflammation of the subject's airway epithelium, of mucus membranes in the subject's nose, of mucus membranes in the subject's eye, of mucus membranes in the subject's gastrointestinal lumen, and/or of the subject's skin.

39. A method according to claim 23, wherein said administering is carried out locally.

40. A method according to claim 23, wherein said administering is carried out locally by administering the compound to the subject's eye, skin, and/or gastrointestinal tract.

41. A method according to claim 23, wherein said administering is carried out locally by administering the compound to the subject's airway.

42. A method according to claim 23, wherein said administering is carried out systemically.

43. A method according to claim 23, wherein the subject is a human.

44. A method according to claim 23, wherein the subject is a human who has previously experienced symptoms of allergic inflammation.

45. A method for reducing a non-animal-derived allergen's ability to initiate an allergenic process, said method comprising:
contacting the non-animal-derived allergen with an agent which inhibits reactive oxygen species.

46. A method according to claim 45, wherein agent is a compound that inhibits the formation of reactive oxygen species.

47. A method according to claim 46, wherein the compound is a NADH/NADPH oxidase inhibitor.

48. A method according to claim 46, wherein the compound is a NADH/NADPH oxidase inhibitor that is selective for NADH/NADPH oxidases.

49. A method according to claim 46, wherein the compound is a NADH/NADPH oxidase inhibitor that is selective for non-animal NADH/NADPH oxidases.

50. A method according to claim 46, wherein the compound is a NADH/NADPH oxidase inhibitor that is selective for pollen and/or mold NADH/NADPH oxidases.

51. A method according to claim 46, wherein the compound is a superoxide dismutase inhibitor.

52. A method according to claim 45, wherein the agent inhibits the activity of reactive oxygen species.

53. A method according to claim 52, wherein the agent is a compound that inhibits the activity of reactive oxygen species.

54. A method according to claim 53, wherein the compound is a scavenger of reactive oxygen species.

55. A method according to claim 54, wherein the compound is a superoxide radical scavenger.

56. A method according to claim 54, wherein the compound is a hydrogen peroxide radical scavenger.

57. A method according to claim 53, wherein the compound is a natural compound.

58. A method according to claim 53, wherein the compound is a synthetic compound.

59. A method according to claim 53, wherein the compound is selected from the group consisting of ascorbic acid, tocopherol, 6-hydroxy-2,5,7,8-tetramethylchroman-2-carboxylic acid, N-acetyl-L-cysteine, plant antioxidants, and combinations thereof.

60. A method according to claim 52, wherein the agent is heat.

61. A method according to claim 45, wherein the non-animal-derived allergen is a pollen.

62. A method according to claim 45, wherein the non-animal-derived allergen is a mold.

63. A method for reducing pro-oxidant activity of a non-animal-derived allergen, said method comprising:
contacting the non-animal-derived allergen with an agent which inhibits reactive oxygen species.

64. A method according to claim 63, wherein agent is a compound that inhibits the formation of reactive oxygen species.

65. A method according to claim 64, wherein the compound is a NADH/NADPH oxidase inhibitor.

66. A method according to claim 64, wherein the compound is a NADH/NADPH oxidase inhibitor that is selective for NADH/NADPH oxidases.

67. A method according to claim 64, wherein the compound is a NADH/NADPH oxidase inhibitor that is selective for non-animal NADH/NADPH oxidases.

68. A method according to claim 64, wherein the compound is a NADH/NADPH oxidase inhibitor that is selective for pollen and/or mold NADH/NADPH oxidases.

69. A method according to claim 64, wherein the compound is a superoxide dismutase inhibitor.

70. A method according to claim 63, wherein the agent inhibits the activity of reactive oxygen species.

71. A method according to claim 70, wherein the agent is a compound that inhibits the activity of reactive oxygen species.

72. A method according to claim 71, wherein the compound is a scavenger of reactive oxygen species.

73. A method according to claim 72, wherein the compound is a superoxide radical scavenger.

74. A method according to claim 72, wherein the compound is a hydrogen peroxide radical scavenger.

75. A method according to claim 71, wherein the compound is a natural compound.

76. A method according to claim 71, wherein the compound is a synthetic compound.

77. A method according to claim 71, wherein the compound is selected from the group consisting of ascorbic acid, tocopherol, 6-hydroxy-2,5,7,8-tetramethylchroman-2-carboxylic acid, N-acetyl-L-cysteine, plant antioxidants, and combinations thereof.

78. A method according to claim 70, wherein the agent is heat.

79. A method according to claim 63, wherein the non-animal-derived allergen is a pollen.

80. A method according to claim 63, wherein the non-animal-derived allergen is a mold.

81. A method for screening a compound for its usefulness as an agent for reducing a non-animal-derived allergen's ability to initiate an allergenic process and/or for its usefulness as a therapeutic agent for treating or preventing allergic inflammation and/or the symptoms associated therewith, said method comprising:
evaluating the compound's ability to inhibit reactive oxygen species.

82. A method according to claim 81, wherein said method is carried out by evaluating the compound's ability to inhibit the formation of reactive oxygen species.

83. A method according to claim 82, wherein said method is carried out by evaluating the compound's ability to inhibit NADH/NADPH oxidase.

84. A method according to claim 82, wherein said method is carried out by evaluating the compound's ability to inhibit pollen and/or mold NADH/NADPH oxidases.

85. A method according to claim 82, wherein said method is carried out by evaluating the compound's ability to inhibit superoxide dismutase.

86. A method according to claim 81, wherein said method is carried out by evaluating the compound's ability to inhibit the activity of reactive oxygen species.

87. A method according to claim 81, wherein said method is carried out by evaluating the compound's ability to scavenge reactive oxygen species.

88. A method according to claim 87, wherein said method is carried out by evaluating the compound's ability to scavenge superoxide radicals.

89. A method according to claim 87, wherein said method is carried out by evaluating the compound's ability to scavenge hydrogen peroxide radicals.
